(19) United States
(12) Patent Application Publication
(10) Pub. No.: US 2013/0330825 A1
(43) Pub. Date: Dec. 12, 2013

(54) ATTACHMENT SUBSTRATES FOR DIRECTED DIFFERENTIATION OF HUMAN EMBRYONIC STEM CELLS IN CULTURE

(71) Applicant: CITY OF HOPE, Duarte, CA (US)

(72) Inventors: Larry A. Couture, Claremont, CA (US); Chang-yi Chen, Arcadia, CA (US)

(21) Appl. No.: 13/802,301

(22) Filed: Mar. 13, 2013

Related U.S. Application Data
(60) Provisional application No. 61/656,952, filed on Jun. 7, 2012.

(51) Int. Cl. C12N 5/071

(52) U.S. Cl. CPC C12N 5/0657
USPC 435/377

(57) ABSTRACT

In one embodiment, methods of producing a population of differentiated target cells from a population of undifferentiated pluripotent stem cells (PSC) are provided herein. Such methods may include culturing the population of undifferentiated PSCs, such as human embryonic stem cells (hESC), on an attachment matrix which comprises at least two or more laminin isoforms. The two or more laminin isoforms may include a laminin combination of one or more laminin isoforms that support the hESC cells; and one or more laminin isoforms that would support a population of differentiated target cells. The one or more laminins (LN) that support the hESC cells may be selected from LN-511 or LN-521, while the population of differentiated target cells is a population of cardiomyocytes, and the one or more laminins that would support the cardiomyocytes are selected from LN-411, LN-111, LN-421, LN-211, LN-332, or LN-121.

### CROSS-REFERENCE TO RELATED APPLICATIONS

This application claims the benefit of U.S Provisional Patent Application No. 61/656,952, filed Jun. 7, 2012 and now pending, which is incorporated herein by reference in its entirety.

### BACKGROUND

The process of directed differentiation of pluripotent stem cells (PSC), such as human embryonic stem cells (e.g. hESC), typically involves a step of culturing and expanding the undifferentiated PSC followed by differentiation induction by adjusting the growth factor composition of the growth medium. These steps are sometimes performed at several points during the process of directed differentiation. Under the adjusted medium conditions, PSCs will differentiate over a period of days to weeks to a desired cell type. This directed differentiation process has been used to produce a variety of cell types including cardiomyocytes, retinal pigment epithelial (RPE) cells, neural stem cells (NSC), dopaminergic neurons, astrocyte progenitor cells, insulin expressing cells, oligodendrocytes, and others.

Currently, several of these hESC culture and differentiation procedures rely on the use of undefined matrices or tissue extracts, such as Matrigel and gelatin, as attachment substrates. Matrigel is a commonly used matrix for production of cardiomyocytes from hESC since it supports both hESC culture as well as differentiated cardiomyocytes. However, Matrigel is produced as an extract from serially transplanted mouse tumors and contains numerous undefined components as well as potentially contaminating mouse viruses and other adventitious agents. Such an undefined matrix is not ideal for GMP manufacturing of cell products used for clinical applications.

Accordingly, a need exists for suitable defined matrices and attachment substrates that support the adherence and growth of desired differentiated cell types, such as cardiomyocytes.

### SUMMARY

In one embodiment, methods of producing a population of differentiated target cells, such as cardiomyocytes, from a population of undifferentiated pluripotent stem cells (PSC) are provided herein. Such methods may include culturing the population of undifferentiated PSCs, such as human embryonic stem cells (hESC), on an attachment matrix which comprises at least two or more laminin isoforms.

The two or more laminin isoforms may include a laminin combination of one or more laminin isoforms that support the hESC cells; and one or more laminin isoforms that support a population of differentiated target cells. In one embodiment, the one or more laminins (LN) that support the hESC cells may be selected from LN-511 or LN-521, while the population of differentiated target cells is a population of cardiomyocytes, and the one or more laminins that would support the cardiomyocytes are selected from LN-411, LN-111, LN-421, LN-211, LN-332, or LN-121. In certain embodiments, the laminin combination may include a combination of LN-511 and LN-411 (411/511); LN-511 and LN-111 (111/511); LN-511 and LN-421 (421/511); LN-511 and LN-211 (211/511); LN-521 and LN-411 (411/521); LN-521 and LN-111 (111/521); LN-521 and LN-421 (421/521); LN-521 and LN-332 (332/521); LN-521 and LN-121 (121/521); or LN-521 and LN-211 (211/521).

Differentiation of the population of undifferentiated PSCs may then be induced by contacting said cells with one or more differentiation factors. In one aspect, the one or more differentiation factors are activin A and BMP-4. In another aspect, the one or more differentiation factors are small molecules including CHIR99021 and IWP4.

### BRIEF DESCRIPTION OF THE DRAWINGS

FIG. 1 is a schematic illustrating cardiac induction of H7 hESCs according to one embodiment. H7 hESCs were plated on 12-well plates coated with a single laminin subtype or a combination of laminin subtypes as shown in Table 1 below. The hESCs were expanded for 5-7 days in Knockout serum replacement (KSR) and two growth factors (2 GFs), bFGF and TGFb. The hESCs were then induced by sequential treatment with Activin A for 1 day in RPMI medium supplemented with B27 (RPMI+B27) followed by treatment with BMP-4 for 4 days in RPMI+B27. The cells were further differentiated in RPMI+B27 for an additional 3-6 days until cells started beating.

FIG. 2A is a set of photographs illustrating the ability of several single laminin (LN) subtypes to support hESC attachment and growth. Single cell-dissociated H7 cells were plated on 12-well plates coated with LN-521, LN-421, LN-411, LN-211, LN-121, or LN-111 (as shown). Only cells plated on LN-521 reached 100% confluence. LN-111 partially supported cell attachment.

FIG. 2B is a set of photographs illustrating the ability of several combinations of laminin (LN) subtypes to support hESC attachment and growth. Single cell-dissociated H7 cells were plated on 12-well plates coated with LN-521 in combination with LN-332 (332/521), LN-421 (421/521), LN-411 (411/521), LN-211 (211/521), LN-121 (121/521), or LN-111 (111/521) as shown. Cell attachment was significantly enhanced when each of the individual subtypes were combined with LN-521.

### DETAILED DESCRIPTION

Methods for producing a population of differentiated target cells from a population of undifferentiated pluripotent stem cells (PSC) are provided herein. Examples of PSCs that may be used in accordance with the embodiments described herein may include, but are not limited to, embryonic stem cells (ESC), embryonic germ cells (ESG), induced pluripotent stem cells (iPSC), embryonic carcinoma cells (ECC), and bone marrow stem cells. The population of differentiated target cells produced from the undifferentiated cells may be any suitable or desired differentiated target cell type including, but not limited to, cardiomyocytes, RPE cells, NSCs, dopaminergic neurons, astrocyte progenitor cells, insulin expressing cells (e.g., pancreatic beta cells) and oligodendrocytes. In one embodiment, the method may be used to produce cardiomyocytes.

According to some embodiments, the methods for producing a population of differentiated target cells, such as cardiomyocytes, may include a step of culturing the population of undifferentiated PSCs on an attachment matrix. In some embodiments, the attachment matrix may be attached to a coated surface (e.g., culture plates or wells) or in a solution to support a suspension culture of PSCs. PSCs are cultured in a set of cell culture conditions that allow the PSCs to be maintained and/or propagated on a coated surface substrate (e.g., a plate or well) or alternatively in a suspension culture on a plurality of substrates (e.g., coated particles or beads). In some embodiments, the cell culture conditions include a culture medium and an attachment matrix. In addition, any suitable culture medium known in the art (e.g., DMEM) may be used in the methods described herein.

The attachment matrix may include one or more attachment substrates. Suitable attachment substrates that may be used in accordance with the methods described herein include Matrigel or one or more extracellular matrix components. The attachment substrate may be one or more defined components or combination of components of Matrigel. In one embodiment, the attachment substrate is a combination of laminin subtypes. Laminins are trimeric proteins having an a chain, a β chain and a γ chain, and contribute to the formation of the basement membrane in the extracellular matrix. At least 15 subtypes (or “isoforms”) of laminin (LN) have been identified, based on their chain composition, including LN-111 (α1β1γ1), LN-121 (α1β2γ1), LN-211 (α2β1γ1), LN-213 (α2β1γ3), LN-221 (α2β2γ1), LN-311 (α3β3γ1), LN-321 (α3β2γ1), LN-332 (α3β3γ2), LN-411 (α4β1γ1), LN-421 (α4β2γ1), LN-423 (α4β2γ3), LN-511 (α5β1γ1), LN-521 (α5β2γ1), LN-522 (α5β2γ2), and LN-523 (α5β2γ3).

Laminins are typically expressed in a tissue-specific manner, thereby supporting the attachment, growth, and differentiation of certain cell types. Presumably because of their tissue specificity, not all laminins support undifferentiated hESC growth and those that do are not necessarily able to support cell adherence or growth of some differentiated cell types, such as cardiomyocytes. As such, laminins have not typically been considered an attractive substrate for hESC growth and differentiation.

As described in the Examples below, laminin isoforms (or “subtypes”) which can support directed differentiation of pluripotent stem cells (PSC), such as human embryonic stem cells (hESC) in adherent culture were identified. In particular, laminin isoforms which can support the production of a population of cardiomyocytes from a population of hESC by directed differentiation were identified in accordance with the embodiments described herein.

According to some embodiments, the attachment substrate includes two or more laminin isoforms. The two or more laminin isoforms may include one or more laminins that support hESC growth in combination with one or more laminins that support a particular population of target cells derived from differentiated hESC. The laminins used in the attachment substrate may also be selected to support any transitional cell types that occur during the differentiation process.

A population of any desired differentiated target cell type may be produced by selecting an appropriate combination of laminin isoforms based on the specific expression profile of the desired differentiated cell type or tissue. The combination of two or more such laminin subtypes is advantageous because the combination is significantly more effective in supporting the differentiation process than the individual laminins on their own, even though each individual laminin may not support the growth of the target cells effectively on its own. In certain embodiments, the one or more laminins that support hESC growth are selected from LN-511 and LN-521 and the one or more laminins that support a population of cardiomyocytes derived from the hESC are selected from LN-411, LN-111, LN-421 and LN-211. In another embodiment, the attachment substrate includes, but is not limited to, a combination of laminin subtypes selected from LN-511 and LN-411 (411/511); LN-511 and LN-111 (111/511); LN-511 and LN-421 (421/511); LN-511 and LN-211 (211/511); LN-521 and LN-411 (411/521); LN-521 and LN-111 (111/521); LN-521 and LN-421 (421/521); and LN-521 and LN-211 (211/521). In some embodiments the attachment may include more than two laminin substrates.

According to certain embodiments, the one or more laminins that support hESC growth may be LN521 and the one or more laminins that support a population of cardiomyocytes derived from the hESC are selected from LN-111, LN-121, LN-211, LN-332, LN-411, and LN-421. In another embodiment, the attachment substrate includes, but is not limited to, a combination of laminin subtypes selected from LN-521 and LN-332 (332/521); LN-521 and LN-411 (411/521); LN-421 (421/521); LN-521 and LN-121 (121/521); LN-521 and LN-211 (211/521); and LN-521 and LN-111 (111/521).

In some embodiments, the combination of two or more laminin subtypes is sufficient for use as the sole substrate during the differentiation process. In other words, no additional substrate components are necessary to support the hESCs, the target cells or any intermediate cells during the differentiation process. In another embodiment, different combinations of laminins may be added or used during different phases of direct differentiation processes. For example, a first combination of one or more laminins may be used to support undifferentiated or “starting” cells, and a second combination of one or more laminins may be used to support differentiated and transitional cells (i.e. an intermediate cell type between the PSC and the final desired differentiated cell type) in cultures where cell passaging is, or could be, practiced.

The cell culture conditions may include one or more additional components to provide a supportive environment for the undifferentiated growth of PSCs or during directed differentiation into the target differentiated cell type (e.g., cardiomyocytes). Examples of additional components may include additional defined substrate components to be included in the attachment matrix, serum (e.g., fetal bovine serum), growth factors and other proteins to be included in the culture medium. For example, suitable substrate components that may be added to the attachment matrix in addition to the laminin isoforms described above include, but are not limited to, polysaccharides, proteins, proteoglycans (e.g., aggrecan, decorin, and heparan), glycoproteins (e.g., vitronectin), glycosaminoglycans (e.g., chondroitin sulfate, dermatan sulfate, heparin sulfate, hyaluronan and keratan sulfate), fibrous proteins (e.g., elastin, keratin, fibronectin, nidogen, and collagen), gelatin, polyomithine or other suitable substrates such as Synthemax®. Suitable growth factors that may be added to the culture medium include, but are not limited to, basic fibroblast growth factor (bFGF), stem cell factor (SCF), fetal liver tyrosine kinase-3 ligand (Flt3L), Noggin (a BMP antagonist), TGFβ1, LIF, nicotinamide (NIC), and keratinocyte growth factor (KGF).

In some aspects, culturing the population of undifferentiated PSCs may additionally include passaging the population of undifferentiated PSCs prior to or during the process of differentiation. The process of passaging the population of cells may be repeated one or more times, and may include dissociating cells supported by the attachment matrix, diluting the dissociated cells in media.

The methods for producing a population of differentiated target cells may, according to some embodiments, additionally include a step of inducing differentiation. Inducing differentiation may be accomplished by changing the composition of growth factors in the growth medium or adding additional growth factors to the growth medium at one or more stages of differentiation. For example, inducing differentiation of PSCs to produce a population of cardiomyocytes may include contacting the cell with one or more differentiation factors including, but not limited to, prostaglandins (e.g., PGI2), minerals (e.g., transferrin, selenium), fibroblast growth factors (e.g., FGF2), insulin-like growth factors (e.g., IGF1), bone morphogenetic proteins (e.g., BMP2, BMP4, and BMP6), Wnt family members, transforming growth factor beta (TGF(3) ligands (e.g., activin A, activin B), platelet-derived growth factor natriuretic factors, insulin, leukemia inhibitory factor (LIF), epidermal growth factor (EGF), vascular endothelial growth factor (VEGF), transforming growth factor alpha (TGFα), and inhibitors for MAP kinase (MAPK), TGFβ, NODAL, BMP, activin, and Wnt. In one embodiment, inducing differentiation of PSCs to produce a population of cardiomyocytes comprises sequential treatment with activin A and BMP-4.

According to certain embodiments, the one or more differentiation factors described above may be one or more small molecules. In one embodiment, the one or more small molecules may modulate Wnt signaling. In some embodiments, the one or more small molecules may be CHIR99021. CHIR99021 modulates Wnt signaling by inhibiting glycogen synthase kinase 3 (Gsk3), which is involved in a number of pathways including differentiation. In certain embodiments, the one or more small molecules may be IWP-4. IWP-4 modulates Wnt signaling by acting as an antagonist in the Wnt/β-catenin pathway. In certain embodiments, inducing differentiation of PSCs to product a population of cardiomyocytes comprises treatment with CHIR99021 and IWP-4.

The differentiated target cells produced using the methods described herein may be cultured for use in methods for screening tissue or cell-specific drugs. For example, a population of cultured cardiomyocytes derived from hESCs may be administered a known or candidate therapeutic to determine its effect on such cells

In another embodiment, differentiated target cells produced according to the methods described herein may be cultured for the purpose of disease modeling.

In certain embodiments, the differentiated target cells produced according to the methods described herein may be administered to a patient as a treatment to replace a population of atrophied cells. The cardiomyocytes produced according to the embodiments described herein may be used in therapeutic methods for improving or restoring cardiac function in a subject who currently has or previously had a cardiovascular disease or condition resulting in the death of a population of cardiomyocytes. Cardiovascular diseases and conditions that are suitable for treatment with cardiomyocytes include, but are not limited to, myocardial infarction, cardiac hypertrophy, congestive heart failure, cardiac arrhythmias, congenital heart disease, cardiomyopathy, or any other condition resulting in the death of cardiomyocytes (e.g., hypertension, coronary artery disease)

The treatment methods described herein may include treating the subject having a population of dead cardiomyocytes as a result of a cardiovascular disease or condition by administering a therapeutically effective amount of a pharmaceutical composition to the subject by any suitable route of administration. In some embodiments, the pharmaceutical composition comprises a population of differentiated cardiomyocytes which were derived from undifferentiated PSCs according to embodiments described above.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

A “therapeutically effective amount,” “therapeutically effective concentration” or “therapeutically effective dose” is the amount of a composition that produces a desired therapeutic effect in a subject, such as preventing or treating a target condition, alleviating symptoms associated with the condition, producing a desired physiological effect, or allowing imaging or diagnosis of a condition that leads to treatment of the disease or condition. The precise therapeutically effective amount is the amount of the composition that will yield the most effective results in terms of efficacy of treatment in a given subject. This amount will vary depending upon a variety of factors, including, but not limited to, the characteristics of the therapeutic composition (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

A “route of administration” may refer to any administration pathway known in the art, including but not limited to aerosol, enteral, nasal, ophthalmic, oral, parenteral, rectal, transdermal, or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In some embodiments, the route of administration may include administering the pharmaceutical composition directly to the site of a target organ or tissue (e.g., infarct area of the heart) by implantation or transplantation. In this case, the administration step may be performed during a surgical procedure or injection. In other embodiments, the route of administration may include administering the pharmaceutical composition according to a cell therapy or gene therapy method.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLE 1

### Use of Specific Laminin Combinations to Support Induction of Pluripotent hESC to Cardiomyocytes

To investigate whether a combination of defined, purified, laminins can replace Matrigel in cardiomyocyte differentiation, different subtypes of recombinant (i.e. purified) laminins were tested with a modified method for hESC cardiomyocyte differentiation. This method was based on a previously described method in which undifferentiated hESCs were first cultured on Matrigel to a high cell density, followed by induction of cardiac differentiation with sequential treatment of Activin A and BMP-4 (see Laflamme et al., Cardiomyocytes derived from human embryonic stem cells in pro-survival factors enhance function of infracted rat hearts, Nature Biotechnology 25:1015-1024 (2007), which is hereby incorporated by reference as if fully set forth herein).

The modified method tested the ability of individual and combinations of laminins to replace Matrigel during the process of hESC cardiomyocyte differentiation. Briefly, single laminin subtypes and combinations of laminin subtypes, shown below in Table 1, were used to coat 12-well plates.

TABLE 1
Single and combinations of laminins used to coat the substrate.

| | | | | | | | | |
|---|---|---|---|---|---|---|---|---|
| single | 511 | 521 | 421 | 411 | 211 | 121 | 111 | |
| combination | | | 421/ | 411/ | 211/ | 121/ | 111/ | 332/521 |
| | | | 521 | 521 | 521 | 521 | 521 | |

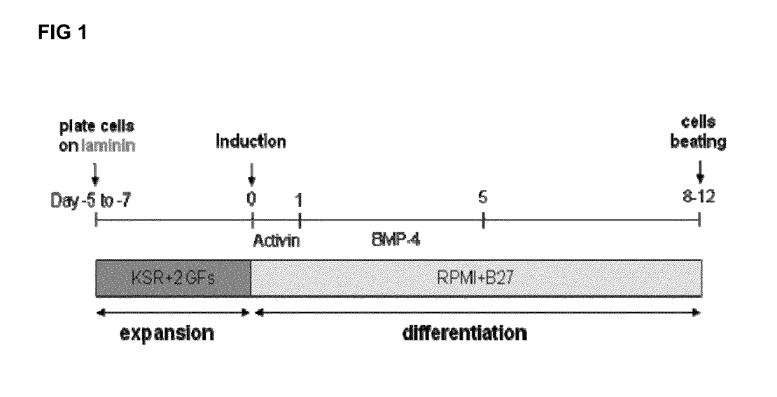

For cardiac differentiation, H7 hESCs were single cells dissociated and plated on the 12-well plates coated with the single laminins or combination as cell attachment matrices at a seeding density of 1.75×10⁵ cells/cm². After 5-7 days of expansion in medium containing Knockout serum replacement (KSR) and two growth factors (GFs), basic fibroblast growth factor (bFGF) and transforming growth factor beta (TGFb), differentiation of the cell cultures was induced with sequential treatment of growth factors: 1 day Activin A and 4 days BMP-4 in RPMI medium supplemented with B27 (FIG. 1). After 5 days of induction, cell cultures were further differentiated in RPMI with B27. Cell beating was observed around day 8 to 12 post Activin A induction. Differentiated cell cultures were harvested at day 19-23 for analysis.

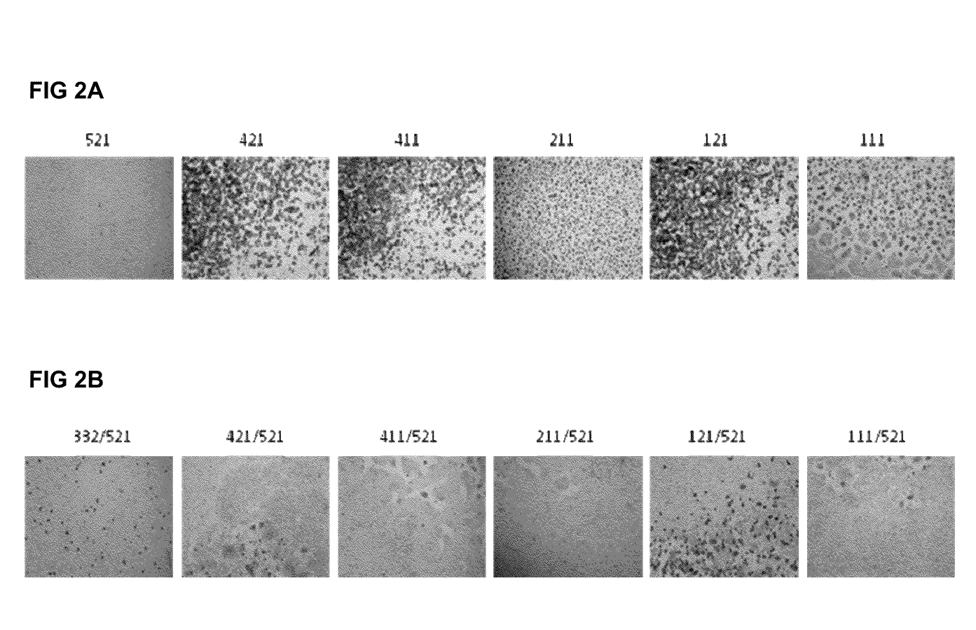

Results showed that some of the individual laminin subtypes (LN-111, LN-121, LN-211, LN-332, LN-411, and LN-421) poorly support undifferentiated hESC attachment as single laminin coatings (FIG. 2A). Because these laminins did not support hESC as single-laminin coatings, their ability to subsequently support cardiomyocytes could not be assessed. In contrast, some of the subtypes tested (LN-511 and LN-521), which were previously reported to support hESC, support hESC attachment and growth, but only poorly support differentiated cardiomyocytes.

To evaluate the effects of subtypes which do not support undifferentiated hESC adhesion on cardiac differentiation, each subtype was individually combined with LN-521 to investigate if they would support or enhance cardiomyocyte differentiation. As shown in FIG. 2B, cell attachment was significantly enhanced when each of the individual subtypes were combined with LN-521. Further, assessment of cardiomyocyte differentiation efficiency and cell yield revealed that several combinations efficiently maintained cell attachment throughout the differentiation process and enhanced cardiomyocyte differentiation yields. The differentiated cells from each of the laminin substrates, alone or in combination (see Table 1), were counted and the cardiac populations were analyzed by flow cytometry for cardiac specific markers, sarcomeric Myosin Heavy Chain (sMHC) and cardiac Troponin T (cTnT). Cell differentiation on Matrigel was used as a control. As shown in Table 2 below, LN-511 or LN-521 alone poorly supported cardiac differentiation from H7 hESCs. As LN-521 was combined with individual laminin subtypes, LN-411, LN-111, LN-421, and LN-211, the cardiac differentiation efficiency was enhanced. LN-111 alone can also support cardiac differentiation. LN-121 or LN-332 combined with LN-521 did not promote cardiac differentiation.

TABLE 2
Efficiency of cardiac differentiation as shown by percentage of cardiomyocytes

| | Matrigel | 511 | 521 | 111 | 411/521 | 111/521 | 421/521 | 211/521 | 121/521 | 332/521 |
|---|---|---|---|---|---|---|---|---|---|---|
| cell | 5.8 × 10⁵ | 7.1 × 10⁵ | 6.5 × 10⁵ | 7.2 × 10⁵ | 9.8 × 10⁵ | 8.6 × 10⁵ | 7.0 × 10⁵ | 5.7 × 10⁵ | 7.8 × 10⁵ | 8.9 × 10⁵ |
| number | | | | | | | | | | |
| sMHC | 41.4% | 26.8% | 20.6% | 36.2% | 30.5% | 44.4% | 36.1% | 42.6% | 26.4% | 20.3% |
| cTnT | 32.4% | 19.7% | 16.1% | 28.7% | 43.1% | 28.0% | 29.1% | 23.0% | 15.2% | 16.9% |

Cardiac cell numbers were calculated with total cell yields and the percentages of sMHC- and cTnT-positive cells, shown below in Table 3. The cardiac populations significantly increased when cells differentiated on the substrate combinations of LN-521 with individual laminin subtypes, LN-411, LN-111, LN-421, and LN-211.

TABLE 3
Cardiac cell yield of cardiomyocytes

| | Matrigel | 511 | 521 | 111 | 411/521 | 111/521 | 421/521 | 211/521 | 121/521 | 332/521 |
|---|---|---|---|---|---|---|---|---|---|---|
| cell | 5.8 × 10⁵ | 7.1 × 10⁵ | 6.5 × 10⁵ | 7.2 × 10⁵ | 9.8 × 10⁵ | 8.6 × 10⁵ | 7.0 × 10⁵ | 5.7 × 10⁵ | 7.8 × 10⁵ | 8.9 × 10⁵ |
| number | | | | | | | | | | |
| sMHC* | 3.3 × 10⁵ | 1.9 × 10⁵ | 1.3 × 10⁵ | 2.6 × 10⁵ | 3.0 × 10⁵ | 3.8 × 10⁵ | 2.5 × 10⁵ | 2.4 × 10⁵ | 2.1 × 10⁵ | 1.8 × 10⁵ |
| cells | | | | | | | | | | |
| cTnT* | 1.9 × 10⁵ | 1.4 × 10⁵ | 1.0 × 10⁵ | 2.1 × 10⁵ | 4.2 × 10⁵ | 2.4 × 10⁵ | 2.0 × 10⁵ | 1.3 × 10⁵ | 1.2 × 10⁵ | 1.5 × 10⁵ |
| cells | | | | | | | | | | |

Taken together with FIG. 2B, the efficiency of supporting cardiomyocyte differentiation is better in some of the laminin isoform combinations. The most efficient combination was the combination of LN-411 and LN-521 (411/521), followed by (in order of efficiency) the combination of LN-111 and LN-521 (111/521), the combination of LN-421 and LN-521 (421/521), and the combination of LN-211 and LN-521 (211/521). In contrast, several combinations of laminins, notably the combination of LN-121 and LN-521 (121/521) and the combination of LN-332 and LN-521 (332/521), failed to enhance the modest activity of the hESC supporting laminin, LN-521. This indicates that some, but not all, of the tissue specific laminins are able to support cardiomyocytes, but that combining a laminin that supports hESC, but only poorly supports cardiomyocytes (e.g. 521), with a laminin that poorly supports hESC, but supports cardiomyocytes, can provide an efficient, defined, substrate to replace Matrigel or other current poorly defined substrates in GMP manufacturing processes.

Although the studies described herein are specific to directed differentiation of hESC to produce cardiomyocytes, similar laminin combinations may be developed and used as a defined matrix for supporting hESC differentiation processes for production of different target differentiated cell types. Further, specific laminin combinations may be developed and used as a defined matrix for the production of cardiomyocytes (or other cell types) from other types of stem or progenitor cells, such as induced pluripotent stem cells (iPSC), bone marrow stem cells, mesenchymal stem cells, umbilical stem cells, and adult cardiac stem cells.

### EXAMPLE 2

### Use of Specific Laminin Combinations to Support Induction of Pluripotent hESC to Cardiomyocytes with Small Molecules

To investigate whether combinations of laminins can also enhance cardiomyocyte differentiation using a different induction method, a modified method was used based on a recent protocol, in which the differentiation was induced by modulating Wnt signaling with small molecules at different differentiation (Lian X. et al., (2012), which is hereby incorporated by reference as if fully set forth herein). Briefly, undifferentiated H7 cells were dissociated into single cells and then were plated at a density of 1,200 cells/cm² on laminin-coated 6-well plates with mTeSR or E8 medium. Medium was changed daily. At day 3 post plating, cells were induced with 12 μM CHIR99021 in RPMI plus B27 for 1 day (the point of induction was defined as day 0). Day 1-3 cells were differentiated with RPMI plus B27. Day 3-5 cells were induced with 5 μM IWP4 in the same media base. After day 5, cells were grown with the media base without addition of any factors. Medium was changed every 2-3 days until cells were harvested at day 18.

As shown in Example 1, LN-521 can support undifferentiated hES cell attachment and growth but poorly supports cardiomyocyte differentiation, while other subtypes LN-111, LN-121, LN-211, LN-332, LN-411, and LN-421 poorly support undifferentiated hES cell attachment. To evaluate whether those laminin subtypes can support or promote cardiomyocyte differentiation using the method of small molecule induction, the individual subtype was combined with LN-521 to coat 6-well plates for cell attachment and differentiation. Coating with LN-521 alone was used as a control.

Results showed that combinations of the individual laminin subtype (LN-111, LN-121, LN-211, LN-332, LN-411, and LN-421) with LN-521 supported cell attachment throughout the differentiation process and enhanced cardiomyocyte differentiation (Table 4). As shown in Table 4, cardiac differentiation on LN-521 generated 21% cTnT positive cells, while the combination of the individual laminin subtype with LN-521 promoted cardiomyocyte differentiation with different efficiency. The combination with the most efficient cardiomyocyte differentiation was the combination of LN-332 and LN-521 (332/521), followed by 411/521, 421/521,121/521, 211/521, and 111/521. In contrast to the Activin A and BMP4 induction method shown in Example 1, LN-332 and LN-121 appeared to be supporting significant cardiac differentiation by induction with small molecules modulating Wnt signaling. Though laminin subtypes support cardiomyocyte differentiation differently with different induction methods, the results from Examples 1 and 2 prove the concept that proper combinations of laminin subtypes enhance cardiomyocyte differentiation.

TABLE 4
Analysis of cardiomycoyte differentiation on different
laminin combinations for cTnT positive cells by flow cytometry

| | Laminin combination | | | | | | |
|---|---|---|---|---|---|---|---|
| | 521 | 111/521 | 121/521 | 211/521 | 332/521 | 411/521 | 421/521 |
| cTnT | 21% | 27% | 37% | 35% | 62% | 45% | 40% |

### REFERENCE

1. Lian X, Hsiao C, Wilson G, Zhu K, Hazeltine L B, Azarin SM, Raval K, Zang J, Kamp T J, and Palecek S P (2012) Robust cardiomyocyte differentiation form human pluripotent stem cells via temporal modulation of canonical Wnt signaling. Proc Natl Acad Sci USA 109(7): E1848-57.

What is claimed is:

1. A method of producing a population of differentiated target cells from a population of undifferentiated pluripotent stem cells (PSC) comprising:
culturing the population of undifferentiated PSCs on an attachment matrix which comprises at least two or more laminin isoforms; and
inducing differentiation of the population of undifferentiated PSCs by contacting said cells with one or more differentiation factors.

2. The method of claim 1, wherein the undifferentiated PSC are human embryonic stem cells (hESC).

3. The method of claim 2, wherein the two or more laminin isoforms comprise a laminin combination of:
one or more laminin isoforms that support the hESC cells; and
one or more laminin isoforms that support a population of differentiated target cells.

4. The method of claim 3, wherein the one or more laminins that support the hESC cells are selected from LN-511 or LN-521.

5. The method of claim 3, wherein the population of differentiated target cells is a population of cardiomyocytes, and the one or more laminins that would support the cardiomyocytes are selected from LN-411, LN-111, LN-421 or LN-211.

6. The method of claim 3, wherein the laminin combination comprises LN-511 and LN-411 (411/511); LN-511 and LN-111 (111/511); LN-511 and LN-421 (421/511); LN-511 and LN-211 (211/511); LN-521 and LN-411 (411/521); LN-521 and LN-111 (111/521); LN-521 and LN-421 (421/521); and LN-521 and LN-211 (211/521).

7. The method of claim 1, wherein the one or more differentiation factors are activin A and BMP-4.

8. The method of claim 3, wherein the one or more laminins that support the hESC cells comprise LN-521.

9. The method of claim 3, wherein the population of differentiated target cells is a population of cardiomyocytes, and the one or more laminins that would support the cardiomyocytes are selected from LN-111, LN-121, LN-211, LN-332, LN-411, or LN-421.

10. The method of claim 3, wherein the laminin combination comprises LN-521 and LN-332 (332/521); LN-521 and LN-411 (411/521); LN-421 (421/521); LN-521 and LN-121 (121/521); LN-521 and LN-211 (211/521); and LN-521 and LN-111 (111/521).

11. The method of claim 1, wherein the one or more differentiation factors are one or more small molecules.

12. The method of claim 11, wherein the one or more small molecules are CHIR99021 and IWP-4.

13. A method of producing a population of cardiomyocytes from a population of undifferentiated human embryonic stem cells (hESC) comprising:
culturing the population of undifferentiated hESCs on an attachment matrix which includes a laminin composition comprising (i) one or more laminin isoforms that support the hESC cells, and (ii) one or more laminin isoforms that would support a population of cardiomyocytes; and
inducing differentiation of the population of undifferentiated hESCs by contacting said cells with one or more differentiation factors.

14. The method of claim 13, wherein the one or more laminins that support the hESC cells are selected from LN-511 or LN-521.

15. The method of claim 13, wherein the one or more laminins that would support the cardiomyocytes are selected from LN-411, LN-111, LN-421 or LN-211.

16. The method of claim 13, wherein the laminin composition comprises LN-511 and LN-411 (411/511); LN-511 and LN-111 (111/511); LN-511 and LN-421 (421/511); LN-511 and LN-211 (211/511); LN-521 and LN-411 (411/521); LN-521 and LN-111 (111/521); LN-521 and LN-421 (421/521); and LN-521 and LN-211 (211/521).

17. The method of claim 13, wherein the one or more laminins that support the hESC cells comprise LN-521.

18. The method of claim 13, wherein the one or more laminins that would support the cardiomyocytes are selected from LN-111, LN-121, LN-211, LN-332, LN-411, or LN-421.

19. The method of claim 13, wherein the laminin combination comprises LN-521 and LN-332 (332/521); LN-521 and LN-411 (411/521); LN-421 (421/521); LN-521 and LN-121 (121/521); LN-521 and LN-211 (211/521); and LN-521 and LN-111 (111/521).

20. The method of claim 13, wherein the one or more differentiation factors are one or more small molecules.
